(19) United States
(12) Patent Application Publication
(10) Pub. No.: US 2010/0206183 A1
(43) Pub. Date: Aug. 19, 2010

(54) Inline antimicrobial additive treatment method and apparatus

(76) Inventor: Rong Yan Murphy, Fayetteville, AR (US)

Correspondence Address: HENRY LAW FIRM;Henry Mark Murphey, P.O. BOX 8850, FAYETTEVILLE, AR, 72703, US

(21) Appl. No.: 12/388,680

(22) Filed: Feb. 19, 2009

(51) Int. Cl. A23L 3/3454

(52) U.S. Cl. 99/487

(57) ABSTRACT

An antimicrobial treatment method for treatment of solid and semisolid foods in industrial food transport systems is provided. For solid and semisolid food applications, the method and related apparatus comprises a conveyor-based transport system in which antimicrobial additives are added to food packaging. The additives are metered into the packages using optical sensors to identify the size of packages, the amount of additive to be administered, and when such packages are in position to receive administration of the additives. The method is capable of realizing greater than 3 log reductions in live microbes in foodstuffs. The technology may also be used to apply any liquid or semi-solid additive or ingredient into packaging, including in nonfood applications such as medical equipment manufacturing.

### CROSS REFERENCES

None.

### GOVERNMENTAL RIGHTS

None.

### BACKGROUND OF THE INVENTION

Undercooked or contaminated foodstuff has caused illness since ancient times. Today, a wide variety of food processing techniques are used to reduce the risk of food-borne illness, and these techniques include the time-honored methods of heating, toxic inhibition (smoking, pickling, etc.), dehydration, low temperature inactivation (freezing) in addition to more modern techniques such as oxidation, osmotic inhibition (use of syrups), freeze drying, vacuum packing, canning, bottling, jellying, heat pasteurization, and irradiation. Generally, such processes do not actually sterilize food, as full sterilization adversely affects the taste and quality of final foodstuffs, but instead reduce microbial content and inhibit further microbial growth. Despite the numerous processes available to food manufacturers to reduce microbes in food, the risk of food-borne illness continues and thus remains the focus of continuing research and development.

Although generally preventable, food-borne illness remains a serious problem to food consumers, government, and industry. Over one-quarter of the population of the United States is affected every year by food-borne illnesses; contaminated food has been estimated by the World Health Organization to cause 76 million illnesses in the U.S. each year, including 325,000 cases resulting in hospitalization and 5,000 deaths. In many cases, microbial contamination occurs during handling when preparing food for retail sale. Although sanitation policies have been improving during recent years, it has proven very difficult to eliminate contamination and pathogens associated with preparing, handling, and processing food at an industrial level. For example, Listeria monocytogenes cannot be eliminated from food or food processing environments using present technologies. A survey by USDA-FSIS showed that between 1% and 10% of retail ready-to-eat deli foods were contaminated with L. monocytogenes. The potential contamination of these and other microbes in foodstuff processing environments presents a serious and continuing food safety threat, which has promoted interest in applying non-heat treatment to foods that kills bacteria and preserves food characteristics. Treating cut fruits and vegetables, seafood, cheese, deli food, meat, poultry, and other foodstuff with non-heat antimicrobial alternatives can reduce or eliminate the presence of microbes.

It is known that more preventative approaches to food safety can reduce or eliminate physical, chemical, and biological hazards in food. “Hazard Analysis and Critical Control Points” (“HACCP”) is a systematic approach to the handling, preparation, and storage of food that aims to prevent food-borne illness at its source rather than inspecting finished products. HACCP works by identifying the steps at which contamination of food is known to occur, and then controlling the environment surrounding food products during those steps, i.e., preventing the entry of contaminants into the sealed processing environment. HACCP is not a process to treat contaminated product; rather, HACCP is a testing methodology to ensure that each step in the process is free from contaminants as well as a strict recording system to verify the results. It is an object of the invention to reduce or eliminate food-borne microbes at virtually any or all stages of an industrial foodstuff processing or preparation system.

The prior art in the field of treating industrial foodstuff to minimize or reduce microbes and contaminants varies widely in form and function, but most references report results measured as the reduction of microbial content between two or more assays in units of “logs,” which represents the difference in microbial content between two assays in terms of orders of magnitude. For example, a commonly sought after and reported goal in the prior art is a reduction by 3 log, which means that the microbial content in a particular sample was reduced by 3 orders of magnitude to 0.1% of its original content. It is thus an object of the invention to utilize an industry standard measurement of effectiveness and to likewise provide for at least a 3 log reduction in microbial content.

Perhaps the oldest approach to eliminating harmful microbes from food is by application of substantial heat. However, the use of heat as an antimicrobial has its drawbacks, including that heat cooks food such that its use is not always appropriate, especially where food is already cooked and is in the process of being packaged. It is an object of the invention to meet or exceed the sanitary achievements surrounding the use of heat while also avoiding the application of heat above that of the ambient temperature at which the food is being processed.

Other more technological methods of destroying microbes on food have been developed in recent years. For instance, U.S. Pat. No. 5,879,732 issued to Caracciolo et al (the '732 patent) discloses a food processing method where animal carcasses are sprayed with an antimicrobial gas/liquid mixture. Because the antimicrobial treatment is gaseous, the treatment must be performed in a chamber that is at least partially enclosed and that has exhaust gas scrubbers to avoid contamination of the processing environment. Furthermore, the gas/liquid mixture is not precisely metered, as evidenced by the fact that the '732 patent requires a drainage pool to capture excess liquid. These drawbacks mean than the '732 patent cannot be retrofitted to industrial conveyor systems of the prior art. It is an object of the invention to provide an antimicrobial treatment device that uses precisely metered liquid to treat foodstuff and which can be retrofitted to preexisting industrial conveyor systems.

U.S. Pat. No. 6,964,788 issued to Phebus et al (the '788 patent) uses a liquid treatment to disinfect foodstuffs whereby cooked foodstuff passes through a clean room on a conveyor and is indiscriminately sprayed with disinfectant, which must be collected and recycled. It is an object of the invention to treat cooked foodstuff with an antimicrobial liquid without the need for a clean room, a collection pool, or a recycling mechanism.

It is a further object of the invention to disclose new methods of non-thermal anti-microbial treatment that hold significant promise for reducing or eliminating microbes from solid and semisolid materials.

Deficiencies of sterilization techniques plague other industries as well, particularly the medical field. Accordingly, it is a further object of the invention to apply to industries in which sterilized items, whether solid or semisolid materials, are desirable.

The apparatus in accordance with the invention provides reliable and relatively inexpensive non-thermal pasteurization and anti-microbial treatment of solid and semisolid materials.

### BRIEF SUMMARY OF THE INVENTION

This antimicrobial treatment process and related apparatus solves many different problems of microbial contaminations in solid and semisolid materials. The processes and apparatuses of this invention can be used with solid and semisolid materials before, during, or after processing or packaging. Specifically, the invention comprises an inline antimicrobial device (IAMD) designed to apply antimicrobial additives to foodstuffs during processing.

Generally, inline manufacturing processing of solid and semisolid food involves a series of conveyors that transport foodstuff at a predetermined velocity and inter-spacing to allow for adequate inspection and packaging. Solid and semisolid foodstuff processing continues uninterrupted until such time as it is desirable to treat the foodstuff with an antimicrobial treatment; in prior art applications using heat, for instance, the inline conveyor system was interrupted to apply batch antimicrobial heat treatments in an oven. In contrast to the prior art, the invention is useful for inline solid and semisolid food conveyor systems in that the invention contemplates antimicrobial treatment as a component of the inline conveyor system rather than a separate, batch-type component.

The invention utilizes an IAMD that applies liquid additives to foodstuffs. As foodstuff moves along a conveyor through the IAMD, an optical sensor detects when foodstuff is moving through the IAMD. The sensor triggers precisely-metered nozzles to spray or drip discrete amounts of antimicrobial additives onto the foodstuff. In contrast, prior art solutions either treated individual packages in a batch process or utilized a continuous spray to treat constantly-moving packages. The former solution wasted time, while the latter wastes antimicrobial additive and also affects the properties of the packaging material and the effectiveness of packaging seals. The invention thus represents an advance in the art due to increased efficiency and improved packaging of industrial food processing systems.

These and other advantages provided by the invention will become apparent from the following detailed description which, when viewed in light of the accompanying drawings, disclose the embodiments of the invention.

### BRIEF DESCRIPTION OF THE DRAWINGS

FIG. 1 is a perspective view of the preferred embodiment of the inline antimicrobial device.

FIG. 2 is a partial top view of the inline antimicrobial device taken along line 2-2 of FIG. 1.

FIG. 3 is a partial side view of the inline antimicrobial device taken along line 3-3 of FIG. 2.

FIG. 4 is a partial side view of the inline antimicrobial device taken along line 4-4 of FIG. 2.

FIG. 5 is a graph showing the movement of packages under the inline antimicrobial device over time.

### LISTING OF COMPONENTS

101—inline antimicrobial device (“IAMD”)
103—conveyor
105—foodstuff
107—frame
109—plumbing system
111—applicator system
113—programmable logic controller (“PLC”)
115—storage tanks
117—tubing
119—housing
121—distributor
123—nozzles
125—conduit
127—nozzle cabling
129—PLC cabling
131—optical sensors
133—optical sensor cabling
135—packaging material

### DETAILED DESCRIPTION OF THE INVENTION

The preferred embodiment of the invention is designed for industrial food processing facilities but may be used in conjunction with standardized processing of materials and products other than foodstuff. The invention comprises an inline antimicrobial device 101 (“IAMD”) that applies precisely metered amounts of fluid additives to foodstuffs moving past the IAMD on a conveyor. IAMD 101 is typically used immediately prior to final product packaging and sealing steps.

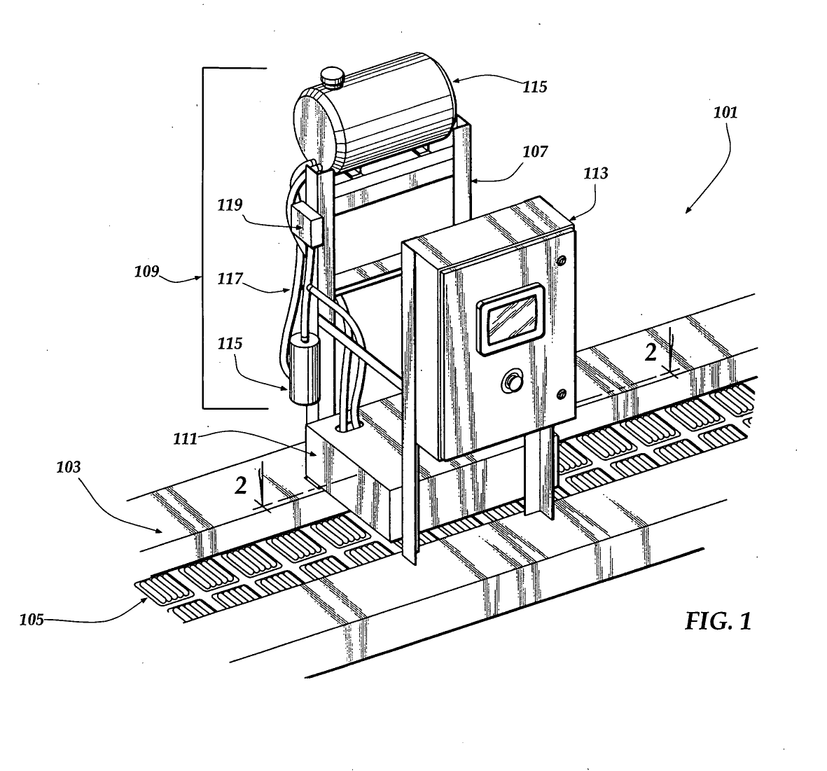

Turning now to FIG. 1, IAMD 101 is designed to work in conjunction with a conveyor 103 that is part of a larger conveyor-based industrial food processing system that moves foodstuff 105 to and through various food processing stations such as IAMD 101. IAMD 101 comprises a frame 107, plumbing system 109, an applicator system 111, and a programmable logic controller 113 (“PLC”).

Plumbing system 109 further comprises one or more storage tanks 115, tubing 117, and one or more regulators 119. Storage tanks 115 may be mounted to frame 107 and serve as a reservoir for the liquid additive that is utilized by applicator system 111. While additive fluid may be pumped from storage tanks 115 to applicator system 111, storage tanks 115 are preferably pressurized in order to provide adequate delivery pressure to applicator system 111. As seen in FIG. 1, more than one storage tank 115 may preferably used to serve as a ballast that assists the regulator in maintaining the pressure in tubing 117 required by applicator system 111. Furthermore, in most industrial applications, two or more storage tanks 115 will be used to store large amounts of additive in a first storage tank 115 at low pressure, which is pumped into a pressurized second storage tank 115.

The preferred pressure for a single storage tank 115 is between 0.1 to 10 psi, or higher depending on the amount of additive fluid to be administered. When more than one storage tank 115 are used, the single storage tank 115 connected to applicator system 111 via tubing 117 is also preferably pressurized to between 0.1 and 10 psi, whereas the pressure of additional storage tanks 115 will vary in relation to their usable volume as compared to the storage tank connected to applicator system 111.

Plumbing system 109 delivers additive fluid, preferably pressurized to between 0.1 and 10 psi, to applicator system 111 through regulator 119 and tubing 117. Regulator 119 lowers the pressure of the additive fluid from the pressure in storage tank 115 to the desired pressure for use in applicator system 111. The greater the difference in pressure between storage tank 115 and the desired pressure for applicator system 111, the higher the rate at which additive fluid 111 may be administered at a relatively constant pressure.

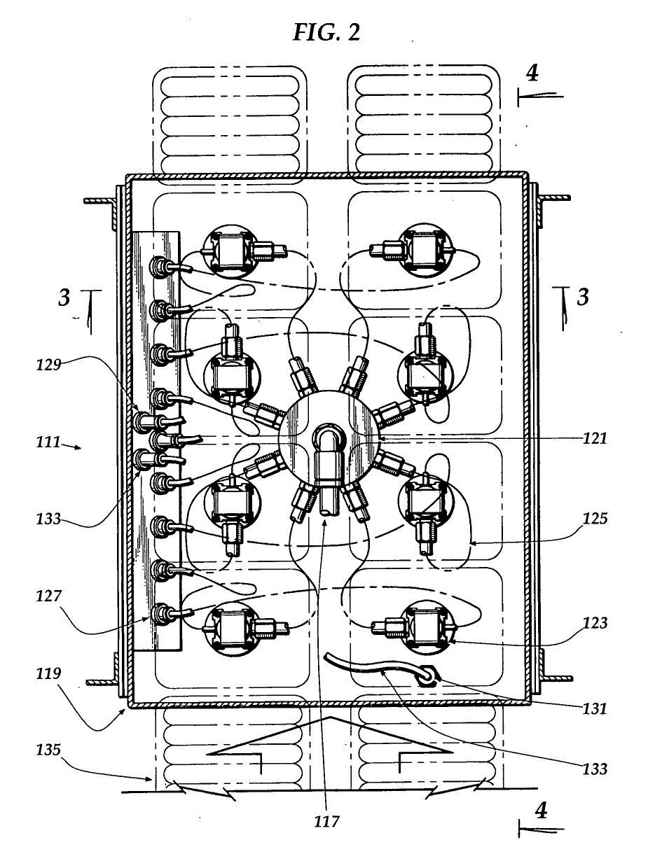

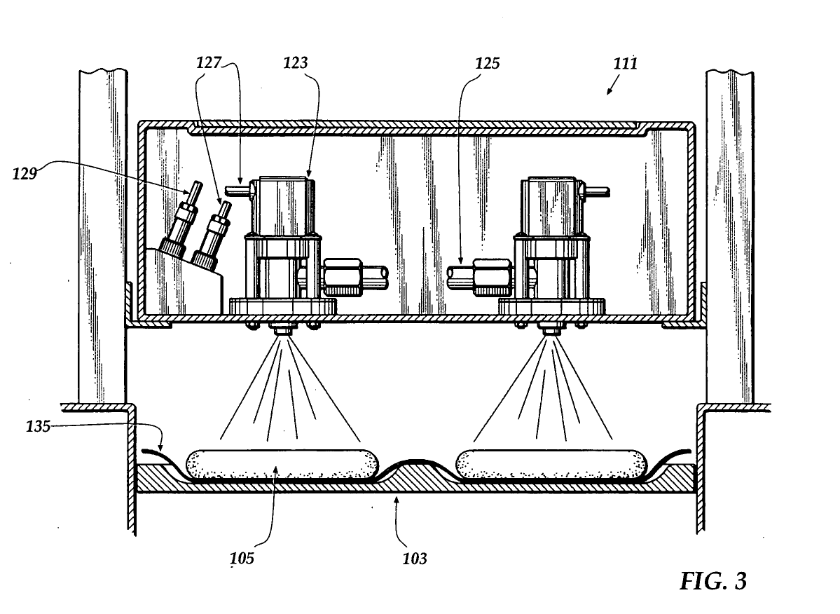

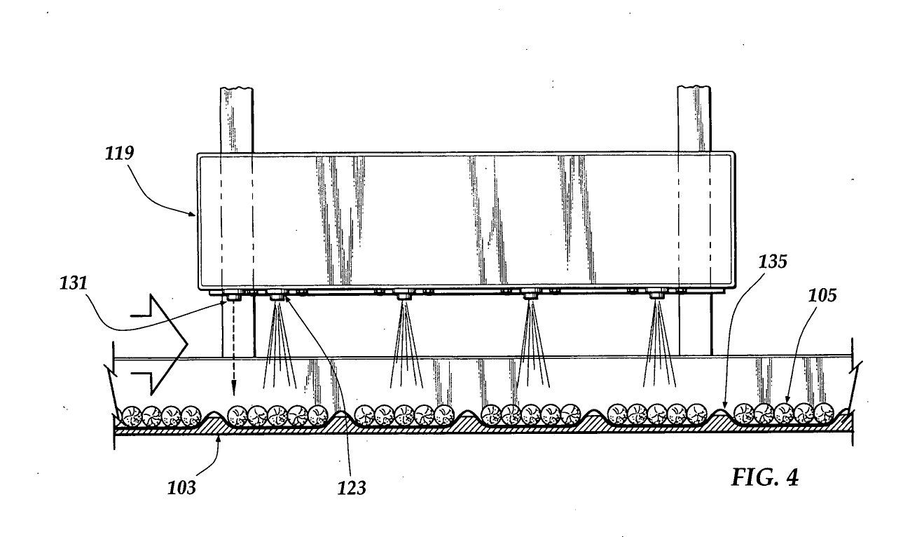

Turning now to FIGS. 2, 3, and 4, applicator system 111 further comprises a housing 119, a distributor 121, one or more nozzles 123, conduit 125, nozzle cabling 127, PLC cabling 129, one or more optical sensors 131, an optical sensor cabling 133. Additive fluid delivered to applicator system 111 enters distributor 121 and is pressure-delivered to nozzles 123 via conduit 125. Nozzles 123 are preferably configured to provide a spray pattern that matches the shape of foodstuff 105 being treated by IAMD 101. Nozzles 123 are controlled by PLC 113, and as such nozzles 123 must be wired to PLC 113 using nozzle cabling 127 and PLC cabling 129. Nozzles 123 preferably have electronically-controlled valves that may be rapidly opened and closed by instructions received from PLC 113. Optical sensors 131 deliver information to PLC 113 via optical sensor cabling 133 about the position of foodstuff 105 on conveyor 103 in relation to applicator system 111.

PLC 113 determines when to administer additive fluid to foodstuff 105 by calculating whether foodstuff 105 is located under one or more nozzles 123. Two factors influence such calculation: first, optical sensor 131 provides a signal to PLC 113 when foodstuff 105 is located substantially beneath optical sensor 131. Preferably, optical sensor can differentiate between packaging material 135 and foodstuff 105. Second, PLC 113 and/or optical sensor 131 determine the linear velocity of conveyor 103. From these two inputs, PLC 113 can determine the location of food with respect to nozzles 123.

As an example of the method by which IAMD 101 applies additive fluid to foodstuff 105, assume that applicator system 111 is approximately 60 cm in length, optical sensors 131 are 3 cm from nearest nozzles 123, nozzles 123 are separated by 15 cm, foodstuff 105 is 10 cm long, and separate articles of foodstuff 105 are separated by 3 cm. The conveyor moves at 50 cm/s. Nozzles are separated into four groups (NG1 to NG4), each group having two nozzles 123 in a line perpendicular to the movement of conveyor 103. Distance refers to the position of the leading edge of foodstuff 105 as compared to optical sensor 131. For instance, a distance of −10 cm means that when the conveyor moves another 10 cm, foodstuff 105 will just be beneath the optical sensor. A distance of 10 cm means that the entire article of foodstuff 105 has just passed beneath optical sensor 131. The following table illustrates when PLC 113 will instruct particular nozzles 123 to turn on to dispense additive fluid while a continuous stream of articles of foodstuff 105 enters IAMD 101:

| t (s) | d (cm) | NG1 | NG2 | NG3 | NG4 |
|---|---|---|---|---|---|
| 0.0 | 0 | off | off | off | off |
| 0.1 | 5 | on | off | off | off |
| 0.2 | 10 | on | off | off | off |
| 0.3 | 15 | off | off | off | off |
| 0.4 | 20 | on | on | off | off |
| 0.5 | 25 | on | on | off | off |
| 0.6 | 30 | on | off | off | off |
| 0.7 | 35 | on | on | on | off |
| 0.8 | 40 | off | on | on | off |
| 0.9 | 45 | on | on | off | off |
| 1.0 | 50 | on | on | on | on |
| 1.1 | 55 | on | off | on | on |
| 1.2 | 60 | on | on | on | off |
| 1.3 | 65 | on | on | on | on |

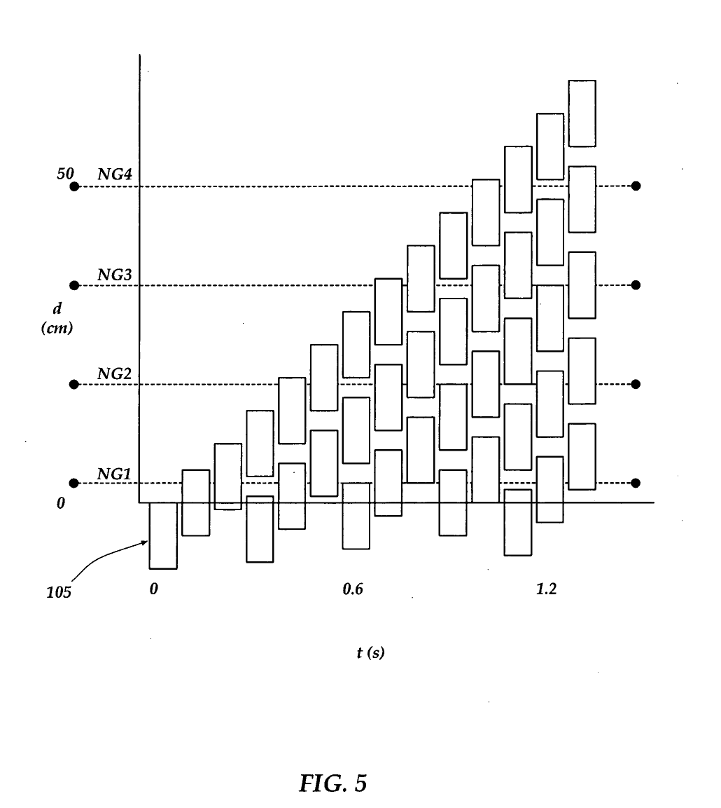

This table is provided to illustrate the role of PLC 113 and is graphically represented in FIG. 5. Persons having ordinary skill in the art will be able to program PLC 113 to perform the functions exemplified in the above table without undue experimentation, and will recognize that such tables are not intended to limit the scope of the invention to the specific dimensions assumed.

The next consideration for PLC 113 is to determine how much additive fluid should be applied to foodstuff 105. Typical food processing lines measure movement in terms of mass per unit velocity (i.e., 100 kg/h @ 3 m/s). Thus, in a typical assembly line, if the velocity of movement is known, the mass is also known. The amount of additive appropriate for any given foodstuff is typically determined by the mass of the foodstuff. Thus, for a given assembly line, the PLC 113 will need to be programmed with the amount of additive to be applied and the mass per unit velocity. Then, the amount of additive fluid to be administered may be calculated from the speed of conveyor 103. For example, when conveyor 103 speeds up, more additive fluid is administered per unit time. PLC 113 may also take into account the pressure, temperature, and viscosity of additive fluid and the flow curves nozzles 123 exhibit under such conditions.

As an example of how PLC 113 determines the amount of additive fluid to apply, assume that the appropriate amount of additive fluid is 10 ml kg, that conveyor 103 moves at 150 cm/s, that each article of foodstuff 105 has a mass of 0.5 kg and is 10 cm long, and that each article of foodstuff will pass under 4 nozzles 123. In this situation, PLC 113 would instruct each of the 4 nozzles 123 to administer 1.25 mL of additive in the 0.067 s it takes for the food to pass under each nozzle, for a total of 5 mL of additive. Persons having ordinary skill in the art will be able to program PLC 113 to perform the functions in the example above without undue experimentation, and will recognize that such example is not intended to limit the scope of the invention to the specific quantities assumed.

The invention may also be utilized in a conveyor-based food processing system in which the movement of conveyor 103 is semi-continuous. For semi-continuous movement of conveyor 103, the movement of conveyor 103 may be defined in terms of index per unit time. The index is defined as number of articles of foodstuff 105 or the length of conveyor moved past a demarcation point, such as optical sensors 131, in one semi-continuous movement of conveyor 103. One semi-continuous movement of conveyor 103 is referred to as an interval. In a semi-continuous embodiment, IAMD 101 applies additive fluid to foodstuff 105 while conveyor 103 is in a stopped position. Preferably, in the semi-continuous method the spray pattern of nozzles 123 match the shape of foodstuff 105 so an even treatment of additive fluid is applied. As discussed above, the amount of additive fluid applied will depend on the mass of foodstuff 105. For example, assume that four articles of foodstuff 105 move past optical sensor 131 in a given interval; IAMD 101 has four nozzles 123; each article of foodstuff has a mass of 0.5 kg; and the appropriate amount of additive fluid is 10 mL/kg. In this situation, each nozzle 123 would release 0.5 mL of additive fluid during each interval.

The invention may be utilized in virtually any conveyor-based processing system. The volumetric capacities of plumbing system 109 and applicator system 111 may be scaled up or down to match the mass per unit velocity required by the particular application. At present, the inventor has realized granularity for application of additive fluid as low as <0.5 mL per cycle of nozzle 123.

The additive fluid contemplated by the invention may be any liquid or semi-solid additive beneficial for use in a conveyor-based system, which may vary depending on the particular application desired by the user. Preferably, the additive fluid comprises a mixture of 5% acetic acid solution, 0.1% propionic acid solution, and 0.1% benzoic acid solution. The additive fluid may also be gaseous, provided that appropriate steps are taken by the user to prevent contamination of the processing environment with the gaseous additive.

While the inventors have described above what they believe to be the preferred embodiments of the invention, persons having ordinary skill in the art will recognize that other and additional changes may be made in conformance with the spirit of the invention and the inventors intend to claim all such changes as may fall within the scope of the invention.

I claim:

1. An inline antimicrobial device for treating foodstuff with antimicrobial additive fluid, comprising:
A programmable logic controller;
A plumbing system having one or more storage tanks, tubing, and one or more regulators;
An applicator system having a distributor, one or more nozzles that receive additive fluid delivered under pressure from the plumbing system, and one or more optical sensors for detecting when foodstuff is beneath the optical sensor, wherein the programmable logic controller calculates when foodstuff is below the nozzles and instructs the nozzles to open when foodstuff is substantially below the nozzles and to close otherwise, thereby precisely metering the additive fluid onto the foodstuff.

2. The inline antimicrobial device of claim 1, wherein the additive fluid comprises a mixture of 5% acetic acid solution, 0.1% propionic acid solution, and 0.1% benzoic acid solution.
